(19) United States
(12) Patent Application Publication
(10) Pub. No.: US 2021/0366617 A1
(43) Pub. Date: Nov. 25, 2021

(54) BIO-ANALYTICS BASED TRIGGERING FOR RECORDING EVENTS

(71) Applicant: Hewlett-Packard Development Company, L.P., Spring, TX (US)

(72) Inventors: Shaun Henry, Boise, ID (US); Gregory Creager, Boise, ID (US); Jonathan Neuneker, Boise, ID (US)

(73) Assignee: Hewlett-Packard Development Company, L.P., Spring, TX (US)

(21) Appl. No.: 17/045,498

(22) PCT Filed: Dec. 5, 2018
(86) PCT No.: PCT/US2018/064033

§ 371 Date: Oct. 6, 2020

(51) Int. Cl. G16H 50/30; G16H 40/67; H04W 4/02

(52) U.S. Cl. CPC G16H 50/30; H04W 4/025; G16H 40/67

(57) ABSTRACT

In an example implementation according to aspects of the present disclosure, a method may receive a biometric datum from a biometric sensor at a normal frequency interval, determine a relationship between the biometric datum and a predetermined threshold based on context which the datum was collected, activate a logging device when the predetermined threshold is surpassed and transmit a notification to a third-party system in the event of an emergency.

### BACKGROUND

Biometric devices and sensors can be utilized for the monitoring of biometric information detected in relation to a user or wearer.

### BRIEF DESCRIPTION OF THE DRAWINGS

FIG. 1 illustrates a system for contextual biometric logging, according to an example;

FIG. 2 is a flow diagram illustrating a method for contextual biometric logging, according to an example;

FIG. 3 is a flow diagram illustrating a method for contextual biometric logging, according to another example of the present disclosure; and

FIG. 4 is a computing device for supporting contextual biometric logging, according to an example.

### DETAILED DESCRIPTION

In one implementation, a biometric sensor may be communicatively coupled to a controller. The controller receives data from the biometric sensor indicative of a state of being of the user or wearer. The controller determines a relationship between the data and a context with which the biometric sensor sampled the data. The relationship may be indicative of normal state of a user within the context or may be indicative of an abnormal state of a user within the context. In an abnormal state, the controller activates a logging device that receives sampling data a higher frequency and notifies a third party system.

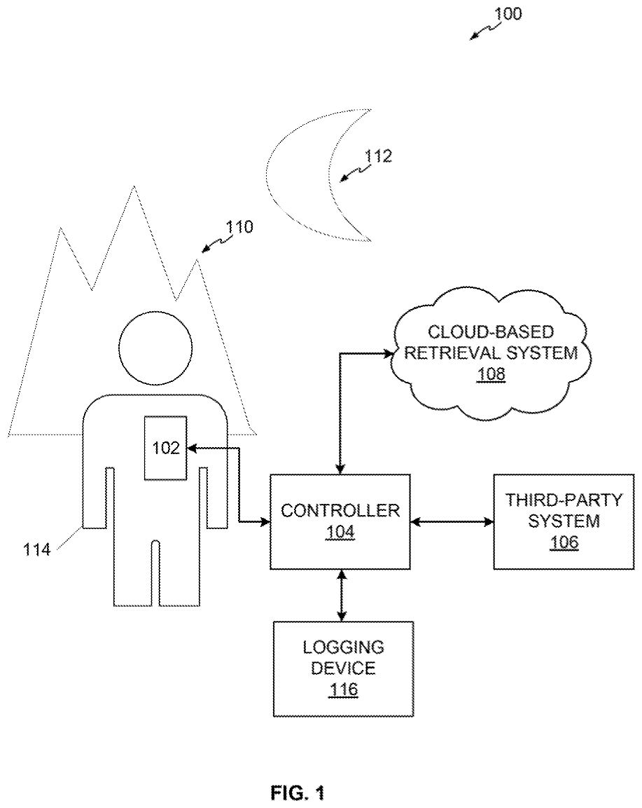

FIG. 1 illustrates a system 100 for contextual biometric logging, according to an example. They system 100 may include a biometric sensor 102, a controller 104, a third-party system 106, a cloud-based retrieval system 108, a geographic location 110, a time of day 112, and a user 114.

The biometric sensor 102 may be a sensor or combination of sensor designed and implemented to monitor biological characteristics of the user. For example, a biometric sensor 102 may include but are not limited to heart rate sensors, perspiration sensors, temperature sensors, and blood pressures sensors. A biometric sensor 102 may be implemented in an internet of things (IoT) form factor as well as using IoT standardized platforms. In another implementation, the biometric sensor 102 may be integrated into a computing device with additional functionality, such as a mobile device, smart phone, or tablet. The biometric sensor 102 samples a designated characteristic for which it is designed at an interval. The interval may be programmatically controlled within the biometric sensor 102, or in another implementation, the biometric sensor 102 may receive external commands to adjust the biometric sensor 102. The interval may have a direct correlation to power consumption of the biometric sensor 102, whereas longer sampling intervals conserve power, and shorter sampling intervals consume more power. The biometric sensor 102 may include communication circuitry to wirelessly connect with external devices. Wireless circuitry may include transceivers that are operable with but not limited to Bluetooth (IEEE 802.15.1), Zigbee (IEEE 802.15.4), near field communication (NFC), ultra-wide band (UWB) (IEEE 802.15.3) and WiFi (IEEE 802.11). The wireless circuitry may allow the biometric sensor 102 to interface and send and receive data with a controller 104.

A controller 104 receives biometric data from the biometric sensor 102. As discussed previously in conjunction with the biometric sensor 102, the controller 104 may include wireless circuitry to allow for wireless communication with the biometric sensor 102. Similar to the biometric sensor 102, the controller 104 may include transceivers that are operable with but not limited to Bluetooth, Zigbee, NFC, UWB and WiFi. In one implementation, the biometric sensor 102 and the controller 104 may be implemented with the same communication protocols. In another implementation, the transceiver may be physically separate from the controller 104, where the controller 104 may access the transceiver functionality through an application programming interface (API). The API may allow the controller 104 to interface with the transceiver and send and receive data to the biometric sensor 102.

The controller 104 may utilize the transceiver to interface with a cloud-based retrieval system 108 and/or a third-party system 106. In one implementation, the controller 104 may utilize a different communication protocol from the protocol utilized in conjunction with the biometric sensor 104. For example, the controller 104 may utilize Bluetooth-enabled communication with the biometric sensor 102 and use UWB with the cloud-based retrieval system 108 and the third-party system 106. Any combination of communication protocol may be implemented by the controller 104 to interface with the biometric sensor 102, cloud-based retrieval system 108, and the third-party system 106.

The controller 104 may include memory to store data biometric data received from the biometric sensor 102. In one implementation, the controller 104 may transmit biometric data from the biometric sensor 102 to the cloud-based retrieval system 108, or the controller 104 may transmit notification to the third-party system 106. When a wireless communication link is not established, the controller 104 may store the biometric data or the notification for delivery to the cloud-based retrieval system 108 or the third-party system 106 when the communication link is reestablished.

The controller 104 may query the biometric sensor 102 for biometric data or, in another implementation, may receive biometric data from the biometric sensor 102 without queries on a set interval schedule. The controller 104 may evaluate biometric data in relation to predetermined thresholds and contexts from which the biometric data was retrieved. The controller 104 may be inclusive to another device with additional functionality such as a mobile device, smart phone or tablet. The controller 104 may be implemented as software, hardware, firmware, or a combination thereof.

A third-party system 106 may receive notifications from the controller 104. The third-party system 106 may include emergency services systems or intermediary safety systems (e.g. OnStar®) (OnStar is a registered trademark of OnStar, L.L.C.).

A cloud-based retrieval system 108 may receive biometric data from the controller 104. The cloud-based retrieval system 108 may collect biometric data and a context corresponding to a specific individual. The biometric data and the context may be stored to create a historical record of the biometric data over time, as well as the context in which the data was collected in. For example, biometric data may be collected during a relaxing beach vacation, as well as a biometric data from a past mountain hiking vacation. In the mentioned examples, the biometric data coupled with the context in which it was captured provide a view into the biological health of the user. A historical data record may provide a detailed view into the health of the user as to what may be considered “normal” and “abnormal” health.

The cloud-based retrieval system 108 may also include advanced machine learning software to evaluate the historical record. The machine learning software may include supervised learning algorithms for classification and regression. The historical record may provide training input to a support vector machine, or other supervised learning algorithm to identify a predetermined threshold for which “abnormal” health may be observed within a context. Additionally, the cloud-based retrieval system 108 may utilize historical record across all users, factoring in the contextual information to determine additional biometric predetermined thresholds when any given user does not have an adequate historical record to support machine learning training.

A context may include but may not be limited to a geographic location 110, a time of day 112, or a combination thereof. The geographic location 110 may be determined using a locational sensor associated with the biometric sensor 102. The geographic location 110 may correspond to a geographic location where the biometric data was collected. The geographic location 110 may be recorded as latitude and longitudinal coordinates in a data package with the biometric data. The time of day 112 may be determined using a digital clock associated with the biometric sensor 102. The time of day 112 may correspond with a time at which the biometric data was collected. The time of day 112 may be recorded as time/date stamp within a data package with the biometric data.

The controller 104 may be communicatively coupled to a logging device 116. In one implementation the logging device 116 may be inclusive to the controller 104, however, in other implementations, the logging device 116 may be physically separate from the controller 104. In implementations where the logging device 116 is separate from the controller 104, the logging device 116 may be able to connect directly with the biometric sensor 102, or alternatively utilize the controller 104 as a proxy. The logging device 116 may store biometric data indicative of an “abnormal” state of a user. The logging device 116 may be able to store the biometric data at a frequency internal higher than normal operation.

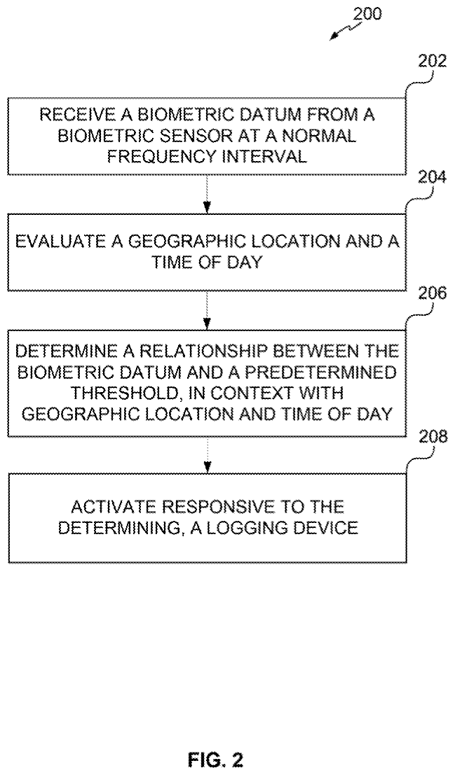

FIG. 2 is a flow diagram 200 illustrating a method for contextual biometric logging, according to an example. At step 202, a controller 104 may receive a biometric datum from a biometric sensor 102 at a normal frequency interval. The biometric datum may be one biometric sample taken at a normal frequency interval. In another implementation the biometric datum may be more than one biometric sample transmitted from the biometric sensor 102 and received by the controller 104 in a single transmission. The normal frequency interval may be set based on an expected power consumption model. The expected power consumption model may provide a balance of samples against battery life of the biometric sensor 102 during normal operation. The controller 104 may receive the biometric datum in the application layer of one of the previously mentioned communication protocols in accordance with the open standards interconnect (OSI) network model.

At step 204, the controller 104 may evaluate a geographic location and a time of day. The controller 104 may extract a geographic location and time of day from the received biometric datum. The controller 104 may parse the application layer of the previously received datum to extract the geographic location and the timestamp corresponding to the biometric datum. In another implementation, in the event that the biometric sensor 102 does not include geographic or timestamping functionality, the controller 104 may append geographic location information and timestamping upon receipt. The appending of the geographic location information and timestamping may approximate a context corresponding to the biometric datum. The controller 104 may query a global positioning system (GPS) receiver to obtain the geographic location. Additionally, the timestamp may also be obtained from GPS geographic location information.

At step 206, the controller 104 may determine a relationship between the biometric datum and a predetermined threshold, in context with geographic location and time of day. The controller 104 parses the biometric datum and compares it to a predetermined threshold. The predetermined threshold may be a number of values stored in a data structure. Each of the values stored in the data structure may correspond to a maximum threshold value for a specific biometric datum. For example, a predetermined value may include values for heart rates corresponding to different times of the day. In the above example, a user may have a historical pattern of having a lowered heart rate during nocturnal hours. The predetermined threshold corresponding to a night time hour may include a lowered heart rate maximum as compared to the predetermined threshold corresponding to a day time hour.

In another example, the predetermined threshold may include a number of values stored in a data structure corresponding to a geographic location. Utilizing the heart rate example discussed above, the stored values in the data structure may correspond to common locations a user is located and historical heart rates corresponding with those locations. In this example, the heart rates in a geographic location of a hiking trail, the predetermined threshold would correspond with a higher maximum value than an office park.

In another example, the predetermined threshold may incorporate both time of day and geographic location as well as more than one biometric sensor thereby creating a multidimensional data structure. Utilizing more than one biometric sensor add a level of confidence to requiring the controller to evaluate predetermined thresholds for all biometric sensors thereby mitigating false positives of malfunctioning biometric sensors.

Upon surpassing a threshold, the controller 104 may query the biometric sensor 102 to provide a higher frequency sampling interval. The higher frequency sampling interval may provide the controller 104 additional data points and granularity corresponding to the biometric the biometric sensor 102 may be monitoring.

At step 208, the controller 104 may activate, responsive to the determining, a logging device 116. The logging device 116 may be inclusive to the controller 104. In one implementation the logging device 116 may be implemented as hardware, software, firmware or any combination thereof. The logging device 116 may receive the higher frequency interval biometric sampling data. The logging device 116 may provide the higher frequency interval data to the controller 104 to provide to the cloud-based retrieval system 108.

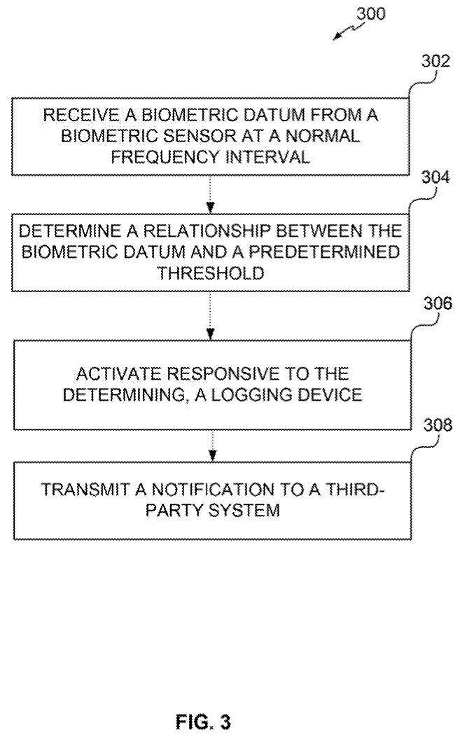

FIG. 3 is a flow diagram 300 illustrating a method for contextual biometric logging, according to another example of the present disclosure. At step 302, the controller 104 may receive a biometric datum from a biometric sensor at a normal frequency interval. As discussed above, the controller 104 may receive biometric datum from the biometric sensor 102.

At step 304, the controller 104 may determine a relationship between the biometric datum and a predetermined threshold. The controller 104 may utilize a context in conjunction with the biometric datum to appropriately compare against the predetermined threshold. A context may be additional information relating to the biometric datum to give transparency into what the datum means. For example, as mentioned above, the context may include geographic location information, or time of day information. Additionally, the context may include but isn't limited to activity information, like exercising, and sleeping information. Additional contexts my better establish predetermined thresholds for those specific activities, as well as providing more historical records to individualize predetermined thresholds for an individual user.

At step 306, the controller 104 may activate responsive to the determining a logging device. As discussed previously, the logging device 116 may be activated upon a biometric datum surpassing a threshold. Prior to the controller 104 determining a biometric datum passes a predetermined threshold, the logging device 116 may be in a low power state. The low power state may be utilized to lower power consumption and prolong battery longevity, as well as conserve storage and memory. In another implementation, the logging device 116 may be toggled on and off via circuitry connected to the controller 104. Upon the activation of the logging device 116, the controller 104 may request and receive a set of biometric data. The set of biometric data may mirror the format of the biometric datum in form. However, the set of biometric data may include one or more additional biometric data presented in a batch. The set of biometric data may be presented to the logging device in a single or multiple batch, or in another implementation, as a stream of one or more biologic datum. In either the batch implementation or the streaming implementation, the sampling rate of the biometric data occurs at a higher frequency interval than the normal frequency interval prior to surpassing the predetermined threshold.

The logging device 116 may be deactivated once the controller 104 detects a biometric datum that falls below the predetermined threshold. In another implementation, the logging device 116 may be deactivated by the controller 104 once a period of time has passed after the biometric data has dropped or does not meet the predetermined threshold.

At step 308, the controller 104 may transmit a notification to a third-party system. The notification may include an indication that the user is in need of help. Additionally, the controller 104 may include a summary of the nature of the help necessary. For example, if the biometric data is indicative of a sudden rise in blood pressure, the notification may include a textual summary of the current blood pressure readings. In another implementation with high bandwidth communication connections between the controller and the third-party system, a log of the event from the passing of the predetermined threshold to current time may be included in the notification. In another implementation, the logging device 116 may transmit the set of biometric data, either in batch or streaming to a cloud-based retrieval system for machine learning training as described previously.

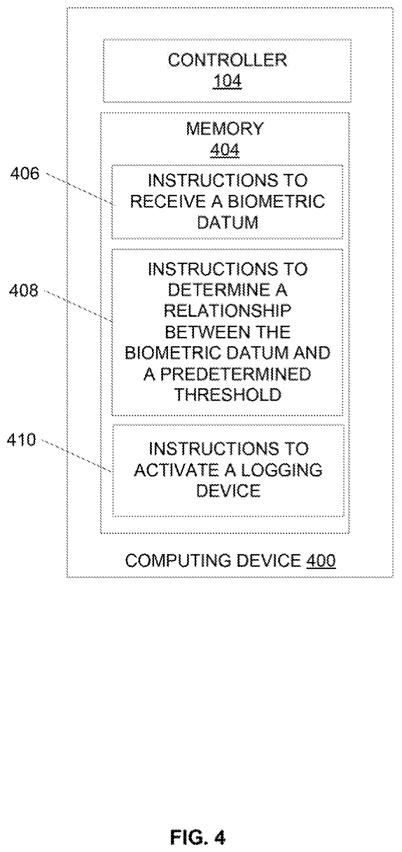

FIG. 4 is a computing device for supporting contextual biometric logging, according to an example. The computing device 400 depicts a controller 104 and a memory device 404 and, as an example of the computing device 400 performing its operations, the memory device 404 may include instructions 406-410 that are executable by the controller 104. The controller 104 may be synonymous with the processor found in common computing environments including but not limited to central processing units (CPUs). The memory device 404 can be said to store program instructions that, when executed by controller 104, implement the components of the computing device 400. The executable program instructions stored in the memory device 404 include, as an example, instructions to receive a biometric datum 406, instructions to determine a relationship between the biometric datum and a predetermined threshold 408, and instructions to activate a logging device 410.

Memory device 404 represents generally any number of memory components capable of storing instructions that can be executed by controller 104. Memory device 404 is non-transitory in the sense that it does not encompass a transitory signal but instead is made up of at least one memory component configured to store the relevant instructions. As a result, the memory device 404 may be a non-transitory computer-readable storage medium. Memory device 404 may be implemented in a single device or distributed across devices. Likewise, controller 104 represents any number of processors capable of executing instructions stored by memory device 404. Controller 104 may be integrated in a single device or distributed across devices. Further, memory device 404 may be fully or partially integrated in the same device as controller 104, or it may be separate but accessible to that device and controller 104.

In one example, the program instructions 406-410 can be part of an installation package that, when installed, can be executed by controller 104 to implement the components of the computing device 400. In this case, memory device 404 may be a portable medium such as a CD, DVD, or flash drive, or a memory maintained by a server from which the installation package can be downloaded and installed. In another example, the program instructions may be part of an application or applications already installed. Here, memory device 404 can include integrated memory such as a hard drive, solid state drive, or the like.

It is appreciated that examples described may include various components and features. It is also appreciated that numerous specific details are set forth to provide a thorough understanding of the examples. However, it is appreciated that the examples may be practiced without limitations to these specific details. In other instances, well known methods and structures may not be described in detail to avoid unnecessarily obscuring the description of the examples. Also, the examples may be used in combination with each other.

Reference in the specification to “an example” or similar language means that a particular feature, structure, or characteristic described in connection with the example is included in at least one example, but not necessarily in other examples. The various instances of the phrase “in one example” or similar phrases in various places in the specification are not necessarily all referring to the same example.

It is appreciated that the previous description of the disclosed examples is provided to enable any person skilled in the art to make or use the present disclosure. Various modifications to these examples will be readily apparent to those skilled in the art, and the generic principles defined herein may be applied to other examples without departing from the scope of the disclosure. Thus, the present disclosure is not intended to be limited to the examples shown herein but is to be accorded the widest scope consistent with the principles and novel features disclosed herein.

What is claimed is:

1. A system comprising:
a biometric sensor;
a controller, communicatively coupled to the biometric sensor, to:
receive a biometric datum from the biometric sensor at a normal frequency interval;
determine a relationship between the biometric datum and a predetermined threshold, wherein the relationship is based at least in part on a context;
activate, responsive to the determining, a logging device.

2. The system of claim 1 wherein the context comprises a geographic location.

3. The system of claim 2 wherein the relationship comprises a comparison between the biometric datum and a predetermined threshold.

4. The system of claim 1 wherein the logging device receives a set of biometric data sampled at a higher frequency interval than the normal frequency interval.

5. The system of claim 1 wherein the logging device transmits the set of biometric data to a cloud-based retrieval system.

6. A method comprising:
receiving a biometric datum from a biometric sensor at a normal frequency interval;
determining a relationship between the biometric datum and a predetermined threshold, wherein the relationship is based at least in part on a context;
activating, responsive to the determining, a logging device; and
transmitting a notification to a third-party system.

7. The method of claim 6 wherein the context comprises a geographic location.

8. The method of claim 7 wherein the relationship comprises a comparison between the biometric datum and a predetermined threshold.

9. The method of claim 6 wherein the logging device receives a set of biometric data sampled at a higher frequency interval than the normal frequency interval.

10. The method of claim 6 wherein the logging device transmits the set of biometric data to a cloud-based retrieval system.

11. A computing device comprising:
a memory having instructions stored thereon and
a processor configured to perform, when executing the instructions to:
receive a biometric datum from a biometric sensor at a normal frequency interval;
determine a relationship between the biometric datum and a predetermined threshold, wherein the relationship is based at least in part on a geographic location of the biometric sensor;
activate, responsive to the determining, a logging device wherein the logging device requests a set of biometric data sampled at a higher frequency interval than the normal frequency interval from the biometric sensor.

12. The computing device of claim 11 wherein the relationship further comprises a time.

13. The computing device of claim 12 wherein the predetermined threshold comprises a historical comparison of a historical biometric datum within the geographic location of the biometric sensor.

14. The computing device of claim 11 wherein the logging device receives a set of biometric data sampled at a higher frequency interval than the normal frequency interval.

15. The computing device of claim 11 wherein the logging device transmits the set of biometric data to a cloud-based retrieval system.
